(19) United States
(12) Patent Application Publication
(10) Pub. No.: US 2024/0165319 A1
(43) Pub. Date: May 23, 2024

(54) System and Method for Capturing Sharps Container Content Information

(71) Applicant: Becton, Dickinson and Company, Franklin Lakes, NJ (US)

(72) Inventor: Ajinkya Madhukar Joglekar, Edgewater, NJ (US)

(21) Appl. No.: 17/992,404

(22) Filed: Nov. 22, 2022

(51) Int. Cl. A61M 5/00; B65F 1/14

(52) U.S. Cl. CPC A61M 5/002; B65F 1/14; A61M 2205/3584; A61M 2205/6054; A61M 2205/6063; B65F 2210/128; B65F 2210/168

(57) ABSTRACT

A medical device disposal container includes a container body defining an opening and at least one sensor proximate the opening, the at least one sensor in communication with a computing device.

### BACKGROUND OF THE INVENTION

### Field of the Invention

The present disclosure relates generally to the disposal of used medical devices.

### Description of Related Art

Used medical devices, particularly those including needles, are commonly disposed of in sharps containers. Many of those used medical devices contain medication and patient information, however, upon disposal in the sharps containers, that information is lost and eventually destroyed. Numerous advantages may be achieved by collecting and analyzing the data 220 stored on those used medical devices, including analytics related to the types of medical devices and medications disposed of in different geographical areas, diversion of medical devices and medications, research and development, and more/

Accordingly, those skilled in the art continue with research and development efforts in the field of disposal and analytics of used medical devices.

### SUMMARY OF THE INVENTION

Disclosed is a medical device disposal container

In one example, the medical device disposal container includes a container body defining an opening and at least one sensor proximate the opening, the at least one sensor in communication with a computing device.

The at least one sensor of medical device disposal container may be configured to capture data stored in an identifier of a used medical device. The at least one sensor may be removably coupled with the container. The at least one sensor may be embedded in the container body. The at least one sensor may be an optical sensor. The at least one sensor may be an RFID reader.

The medical device disposal container may include a lid coupled with the opening. The at least one sensor may be located on the lid.

Also disclosed is a system for aggregating disposable medical device data.

In one example, the system includes a computing device and a medical disposal container in communication with the computing device. The medical device disposal container includes a container body defining an opening and at least one sensor proximate the opening.

The at least one sensor of the system may be in wireless communication with the computing device. The computing device may include a memory, a processor, a bus, and a storage component.

The system may further include at least one used medical device 160 having at least one identifier. The at least one used medical device may be a syringe.

Also disclosed is a method for aggregating disposable medical device data.

In one example, the method includes sensing data with a sensor and transmitting the data to a computing device.

The sensing may be performed optically and/or wirelessly. The transmitting may be performed wirelessly. The method may further include receiving a used medical device and analyzing the data after the sensing.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective schematic of a medical device disposal container;

FIG. 2 is a block diagram of a system for aggregating disposable medical device data;

FIG. 3 is a schematic of the system of FIG. 2; and

FIG. 4 is a flow diagram of a method for aggregating disposable medical device data.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

As used herein, the terms “communication” and “communicate” refer to the receipt or transfer of one or more signals, messages, commands, or other type of data 220. For one unit (e.g., any device, system, or component thereof) to be in communication with another unit means that the one unit is able to directly or indirectly receive data 220 from and/or transmit data 220 to the other unit. This may refer to a direct or indirect connection that is wired and/or wireless in nature. Additionally, two units may be in communication with each other even though the data 220 transmitted may be modified, processed, relayed, and/or routed between the first and second unit. For example, a first unit may be in communication with a second unit even though the first unit passively receives data 220 and does not actively transmit data 220 to the second unit. As another example, a first unit may be in communication with a second unit if an intermediary unit processes data 220 from one unit and transmits processed data 220 to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data 220 from the client. An “interface” refers to a generated display, such as one or more graphical user interfaces (GUIs) with which a user may interact, either directly or indirectly (e.g., through a keyboard, mouse, touchscreen, etc.).

As used herein, the term “server” may refer to or include one or more processors or computers, storage devices, or similar computer arrangements that are operated by or facilitate communication and processing for multiple parties in a network environment, such as the Internet, although it will be appreciated that communication may be facilitated over one or more public or private network environments and that various other arrangements are possible. Further, multiple computers, e.g., servers, or other computerized devices, such as POS devices, directly or indirectly communicating in the network environment may constitute a “system,” such as a merchant's POS system.

This disclosed medical device disposal container 100, system 200, and method 300 are designed to integrate a sensor 150 to capture medication information on a used medical device 160, such as a syringe, prior to disposal. The method 300 to capture the medication information could utilize an active sensor (e.g. optical) that reads the information printed on the label adhered to the syringe. Alternatively, it could be a passive sensor (e.g. RFID/NFC chip) that is embedded in the syringe and contains medication information. In either case the information is read when the syringe is disposed. In addition to the medication information (e.g. drug name, dose, and concentration) other information on the label or chip, such as diluent used or message alerts, could be transmitted as well. The information captured can be stored locally or can be sent to a server via wireless connectivity. Usually, sharps containers hung on the wall or placed on a surface and are in close proximity to a wall outlet that the container could also be plugged into for its power needs.

The information and data collected may be further aggregated and analyzed to build analytics like most common drug prescribed/injected, quantify site carbon footprint etc. Further, the information can be used to help with addressing drug diversion. It is further contemplated that the disclosed medical device disposal container, system, and method are configured to integrate with Intelliport™ to map the med syringe journey as well as data from it could be integrated into the SiteScan™ dashboard.

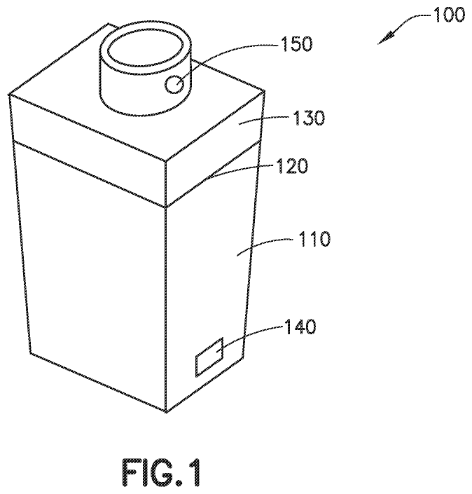

Referring to FIG. 1, disclosed is a medical device disposal container 100. The medical device disposal container 100 may be for receiving used syringes or other medical devices having sharps. For example, the medical device disposal container 100 may be a sharps container. The medical device disposal container 100 includes a container body 110. The container body 110 defines an opening 120. The opening 120 is sized and shaped to receive a used medical device 160, such as used syringes or other medical devices with needles.

Still referring to FIG. 1, the medical device disposal container 100 includes at least one sensor 150 located proximate the opening 120. The at least one sensor 150 may be embedded in the container body 110 such that they are a single, monolithic piece, or may be removably coupled with the container body 110. The at least one sensor 150 may be positioned on the container body 110 such that when a used medical device 160 is disposed of in the container 100 via the opening 120, the at least one sensor 150 can sense and/or read information stored in an identifier 170 located on the used medical device 160.

The at least one sensor 150 is configured to be in communication with a computing device 210. In one example, the at least one sensor 150 is in wireless communication with the computing device 210. In another example, the at least one sensor 150 includes a remote computing device 152 configured to transmit and/or process data 220. The at least one sensor 150 and computing device 210 may be in communication via any suitable means of wireless transmission, such as but not limited to Wi-Fi, cellular network, Bluetooth, radio transmission, or any other suitable means.

The at least one sensor 150 is configured to sense, collect, and transmit data 220 stored on a used medical device 160, such as a syringe. The used medical device 160 may include information stored on it in the form of a barcode, quick response (QR) code, active radio-frequency identification (RFID) tag, passive RFID tag, near field communication (NFC) chip, or other means of storing data 220 related to the medication and/or patient who was prescribed the medication.

As stated above, the RFID tags may be passive tags, which have no power source and are powered by energy received wirelessly from the RFID reader, or active tags, which are powered by a local or internal power source (e.g., a battery, etc.). The RFID tags may include an antenna and a device to electronically store information and/or data 220 to be read from the RFID tags by the RFID reader (e.g., a memory chip, etc.).

RFID typically operates in three main frequency ranges: Low Frequency (LF) (e.g., about 120-150 KHz), High Frequency (HF) (e.g., about 13.56 MHz), and Ultra-High Frequency (UHF) (e.g., about 865-868 MHz in Europe and 902-928 MHz in the U.S.). The LF and HF frequency ranges use magnetically coupled systems where the fields of the RFID reader antenna and the fields of the RFID tag must line up for the RFID reader to read the RFID tag. The LF and HF frequency ranges work well if the orientation of the RFID tags with respect to the reader can be controlled. In contrast, in the UHF frequency range, the RFID tags may still be read if the field orientation of the RFID tags is not directly lined up with the RFID reader antenna. Accordingly, UHF RFID tags may perform better in situations in which the orientation of the RFID tags with respect to the RFID reader cannot be easily controlled and/or known.

Accordingly, the at least one sensor 150 of the medical device disposal container 100 may be any type needed for the intended application. In one example, the at least one sensor 150 is an optical sensor configured to optically capture data 220, such as from a bar code or QR code. In another example, the at least one sensor 150 is an NFC reader or RFID reader configured to capture data 220 from an identifier 170, the identifier 170 being one or more NFC chip RFID tag, respectively. The reader is configured to communicate wirelessly with NFC chips and/or RFID tags.

Still referring to FIG. 1, in one or more examples, the medical device disposal container 100 may further include a lid 130. The lid 130 may cover the opening 120 of the medical device disposal container 100. In one example, the at least one senor 150 is located on the lid 130 of the medical device disposal container 100.

The medical device disposal container 100 may further include a socket 140 for coupling with a power source, such as an electrical outlet. The socket 140 may be used to power the at least one sensor 150.

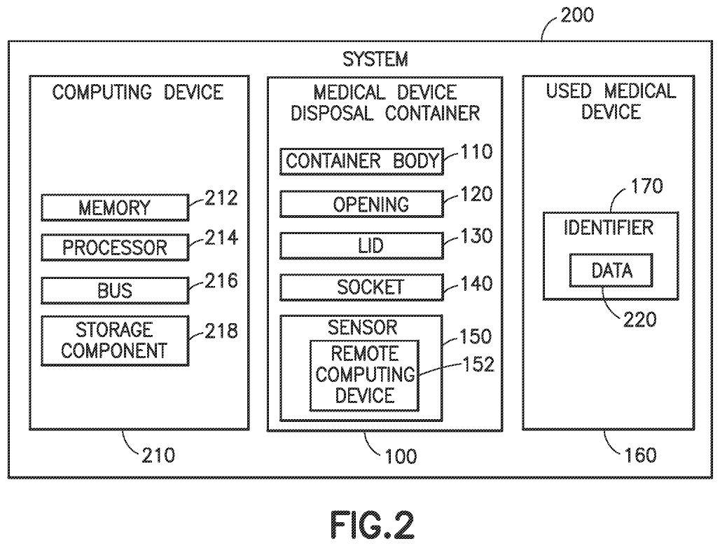

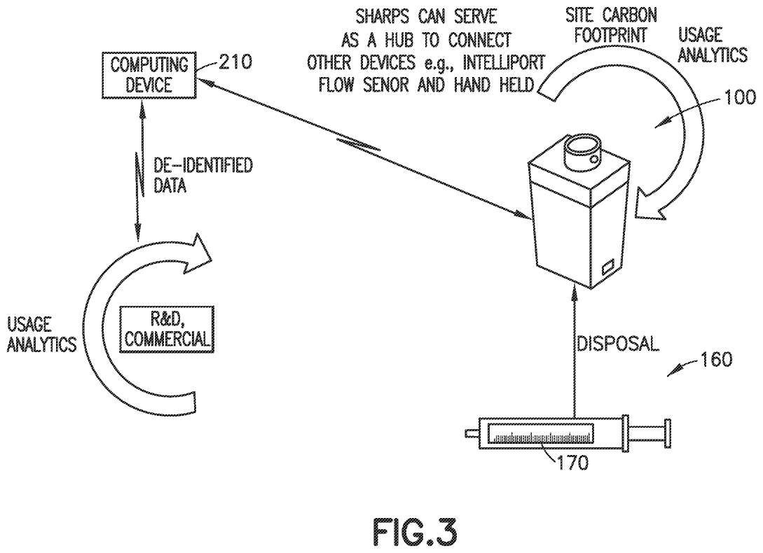

Referring to FIG. 2 and FIG. 3, in one or more examples, disclosed is a system 200 for aggregating disposable medical device 160 data 220.

Still referring to FIG. 2 and FIG. 3, the system 200 includes a medical device disposal container 100 as shown and described herein. The medical device disposal container 100 includes a container body 110 that defines an opening 120. The medical device disposal container 100 further includes at least one sensor 150 located proximate the opening 120.

Still referring to FIG. 2 and FIG. 3, the system 200 includes a computing device 210 in communication with the at least one sensor 150. In one example, the computing device 210 is in wireless communication with the at least one sensor 150 on the medical device disposal container 100. The computing device 210 may include a memory 212, processor 214, bus 216, and a storage component 218. The computing device 210 may be configured to receive, store, analyze, and transmit data 220 received from the at least one sensor 150.

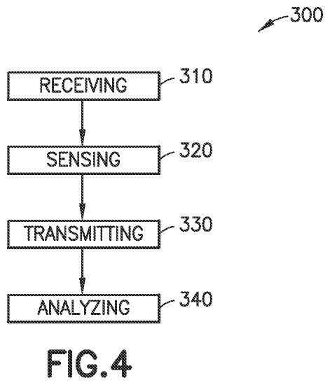

Referring to FIG. 4, also disclosed is a method 300 for aggregating disposable medical device 160 data 220. The method 300 initially includes receiving 310 a used medical device 160. The receiving 310 may be performed when a user disposes of a used medical device 160 in a medical device disposal container 100 through the opening 120.

The method 300 includes sensing 320 data 220 with a sensor 150. The data 220 may be stored in an identifier 170 located on the used medical device 160. The identifier 170 may be any one of a barcode, NFC chip, RFID tag, QR code, or other means of storing data 220. In one example, the sensing 320 is performed optically. In another example, the sensing 320 is performed wirelessly.

Still referring to FIG. 4, in one or more examples, the method 300 includes transmitting 330 the data 220 to a computing device 210. In one example, the transmitting 330 is performed wirelessly. The method 300 may further include analyzing 340 the data 220 after the sensing 320. The analyzing 340 may be performed by the computing device 210, or may be performed manually by analysts who receive the data 220 from the computing device 210.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A medical device disposal container comprising:
a container body defining an opening; and
at least one sensor proximate the opening, the at least one sensor in communication with a computing device.

2. The medical device disposal container of claim 1, wherein the at least one sensor is configured to capture data stored in an identifier of a used medical device.

3. The medical device disposal container of claim 1, wherein the at least one sensor removably coupled with the container body.

4. The medical device disposal container of claim 1, wherein the at least one sensor is embedded in the container body.

5. The medical device disposal container of claim 1, wherein the at least one sensor is an optical sensor.

6. The medical device disposal container of claim 1, wherein the at least one sensor is an RFID reader.

7. The medical device disposal container of claim 1, further comprising a lid coupled with the opening.

8. The medical device disposal container of claim 7, wherein the at least one sensor is located on the lid.

9. A system for aggregating disposable medical device data, the system comprising:
a computing device; and
a medical device disposal container in communication with the computing device, the medical device disposal container comprising;
a container body defining an opening; and
at least one sensor proximate the opening.

10. The system of claim 9, wherein the at least one sensor is in wireless communication with the computing device.

11. The system of claim 9, wherein the medical device disposal container comprises a lid coupled with the opening of the container body.

12. The system of claim 9, wherein the computing device comprises:
a memory;
a processor;
a bus; and
a storage component.

13. The system of claim 9, further comprising at least one used medical device having at least one identifier.

14. The system of claim 13, wherein the at least one used medical device is a syringe.

15. A method for aggregating disposable medical device data, the method comprising:
sensing data with a sensor;
transmitting the data to a computing device.

16. The method of claim 15, wherein the sensing is performed optically.

17. The method of claim 15, wherein the sensing is performed wirelessly.

18. The method of claim 15, wherein the transmitting is performed wirelessly.

19. The method of claim 15, further comprising receiving a used medical device.

20. The method of claim 15, further comprising analyzing the data after the sensing.
